(19) United States
(12) Patent Application Publication
(10) Pub. No.: US 2013/0310304 A1
(43) Pub. Date: Nov. 21, 2013

(54) COMPOSITIONS AND METHODS FOR THE TREATMENT OF ATHEROSCLEROSIS

(71) Applicants: Matthew C. Lawyer, Park City, UT (US); Carl Lawyer, Poplar Bluff, MO (US)

(72) Inventors: Matthew C. Lawyer, Park City, UT (US); Carl Lawyer, Poplar Bluff, MO (US)

(21) Appl. No.: 13/704,477

(22) PCT Filed: Jun. 15, 2011
(86) PCT No.: PCT/US2011/040544

§ 371 Date: Aug. 5, 2013

Related U.S. Application Data
(60) Provisional application No. 61/354,743, filed on Jun. 15, 2010.

(51) Int. Cl. C07K 14/47

(52) U.S. Cl. CPC C07K 14/47
USPC 514/1.9; 530/350; 536/23.5; 435/320.1

(57) ABSTRACT

Novel HDL mimetic polypeptides are disclosed, particularly Apo A-1 Carnivora/Sapien polypeptides. Nucleic acids coding for the Apo A-1 Carnivora/Sapien polypeptides are also disclosed, as are pharmaceutical formulations comprising the disclosed polypeptides and pharmaceutical formulations comprising the disclosed nucleic acids. Finally, methods to prevent, treat, ameliorate, and diagnose atherosclerosis and related disorders, in mammals, such as humans, are disclosed.

### FIELD OF INVENTION

The present invention is directed to the fields of cardiovascular medicine and cell biology of the cardiovascular system. Various embodiments relate generally to methods and compositions involving novel apolipoprotein A-1 Carnivora/Sapien (“A1 CS”) polypeptides for the treatment, amelioration, diagnosis, or prevention of atherosclerosis and related disorders.

### DETAILED DESCRIPTION

The present invention provides purified and isolated polynucleotide molecules that encode novel HDL mimetic polypeptides, such as Apo A-1 Carnivora/Sapien polypeptides, which can be used in methods to prevent, treat, ameliorate, and diagnose atherosclerosis and related disorders, in mammals, such as humans.

The invention further encompasses doses and dosing regimens for the use of Apo A-1 Carnivora/Sapien and pharmaceutical formulations of Apo A-1 Carnivora/Sapien to treat or prevent atherosclerosis and related disorders, including acute coronary syndromes, ischemia, ischemic reperfusion injury, angina and myocardial infarction and the reduction or stabilization of atherosclerotic plaque, the reduction of plaque in occluded vessels and promotion of cholesterol efflux.

The invention further encompasses doses and dosing regimens for the use of Apo A-1 Carnivora/Sapien:phospholipid complex and pharmaceutical formulations of Apo A-1 Carnivora/Sapien:phospholipid complex to treat or prevent cardiovascular disease or related disorders including atherosclerosis, acute coronary syndromes, ischemic reperfusion injury, angina and myocardial infarction, and the reduction or stabilization of atherosclerotic plaque, the reduction of plaque in occluded vessels and promotion of cholesterol efflux.

One form of the invention provides polypeptides corresponding to the isolated DNA molecules. In certain embodiments, the amino acid sequences of the corresponding encoded polypeptides are shown for Apo A-1 Carnivora/Sapien polypeptide 1 as SEQ ID NO: 2.

Those skilled in the art will readily understand that the invention is understood to provide derivatives of such polypeptides and fragments derived therefrom, which result from the addition, deletion, or substitution of non-essential amino acids as described herein.

The present invention further provides a method for producing a polypeptide of the invention in a recombinant host system and related expression cassettes, vectors, and transformed or transfected cells.

The present invention further provides therapeutic and/or prophylactic methods of using of a polypeptide of the invention, either in a naked form or formulated with a delivery vehicle, and related pharmaceutical compositions.

In other embodiments of the invention, a kit is included with a dosage form of a polypeptide of the invention and instructions for its use in prevention or treatment of atherosclerosis in a mammal, such as accompanying labeling to identify the formulation contained therein and other information useful to health care providers and subjects in the prevention or treatment of atherosclerosis, including, but not limited to, instructions for use, dose, dosing interval, duration, indication, contradictions, warnings, precautions, handling and storage instructions and the like.

The present invention further provides a recombinant adeno-associated viral (rAAV) vector and delivering the rAAV vector to a mammal. The invention further includes transducing the rAAV vector encoding an Apolipoprotein A-1 Carnivora/Sapien (A1 CS). The invention includes a purified rAAV vector encoding A1 CS, and a host cell genetically modified with rAAV-A1 CS. In another embodiment of the invention, the rAAV-A1 CS vector is first transduced into multipotent stem cells. The invention further includes transplanting the multipotent stem cells into a mammal. The multipotent stem cells may be bone marrow, cord blood or cytokine-primed peripheral blood cells.

In various embodiments of the invention, a kit is included with the rAAV vector of the invention and instructions for its use in the prevention or treatment of atherosclerosis in a mammal.

Methods, compositions and dosage regimens are provided herein, and are believed to encompass safe and effective treatments, without being limited by theory, that rapidly promote cholesterol efflux and mobilization from lipidic plaques in the arteries, which thereby confer benefit in terms of improvement of endothelial function, prevention of endothelial dysfunction, or improvement or prevention of atheromatous plaques that can rupture and lead to thrombotic events, such as a myocardial infarction. The mechanism of action encompasses improved blood flow or perfusion of the arteries that directly or indirectly confers improvement in endothelial function, or prevention of endothelial dysfunction, or confers rapid reduction or stabilization of unstable plaques.

Consensus amino acids have been identified in amino acid sequences that correspond to Apolipoprotein A-1 polypeptides of some animals of the Carnivora order (e.g., dogs), which display atherosclerosis resistance. The consensus amino acids have been further identified as contrasting with amino acids found at the same location in amino acid sequences corresponding with wild-type human Apolipoprotein A-1. Synthetic Apolipoprotein A-1 polypeptides, such as Apo A-1 Carnivora/Sapien Polypeptide 1, comprised of the amino acid sequence corresponding with wild-type human Apolipoprotein A-1 modified by substitutions of one or more of these identified Carnivora consensus amino acids can be used to prevent and treat atherosclerosis disease.

The Apo A-1 Carnivora/Sapien Polypeptide 1, whose 242 amino acid residue sequence is shown as SEQ ID NO: 2, is an approximately 28 kDa size protein.

The wild-type human Apo A-1 preprotein amino acid sequence is set forth in SEQ ID No: 1 shown below. The first eighteen amino acids form the signal peptide and the next six amino acids form the propeptide.

(SEQ ID NO: 1)
MKAAVLTLAVLFLTGSQARHFWQQDEPPQSPWDRVKDLATVYVDVL
KDSGRDYVSQFEGSALGKQLNLKLLDNWDSVTSTFSKLREQLGPVT
QEFWDNLEKETEGLRQEMSKDLEEVKAKVQPYLDDFQKKWQEEMEL
YRQKVEPLRAELQEGARQKLHELQEKLSPLGEEMRDRARAHVDALR
THLAPYSDELRQRLAARLEALKENGGARLAEYHAKATEHLSTLSEK
AKPALEDLRQGLLPVLESFKVSFLSALEEYTKKLNTQ

The synthetic Apo A-1 Carnivora/Sapien Polypeptide 1 amino acid sequence is set forth in SEQ ID NO: 2 shown below.

(SEQ ID NO: 2)
DEPPQSPWDRVKDLATVYVDVLKDSGRDYVSQFEGSALGKQLNLKL
LDNWDSVTSTFSKLREQLGPVTQEFWDNLEKETEGLRQEMSKDLEE
VKAKVQPYLDDFQKKWQEEMELYRQKVEPLRAELQEGARQKLHELQ
EKLSPLGEEMRDRARAHVDALRTHLAPYSDELRERLAARLEALKEN
GGARLAEYHAKATEHLSTLSEKAKPALEDLRQGLLPVLESFKVSFL
SALEEYTKKLNT

For the sake of consistency, the location of amino acids in all amino acid sequences discussed herein maintain the nomenclature of the location of amino acids in the sequence corresponding with the wild-type human Apo A-1 preprotein (SEQ ID NO: 1). Thus, the first amino acid in SEQ ID NO: 2 (i.e., “D”) is denoted as having the location “#25” in the amino acid sequence and may, for example, be accurately referred to as “D at #25” or “Asp25”.

The Apo A-1 Carnivora/Sapien Polypeptide 1 is identified as a novel variant of wild-type human Apo A-1 by its amino acid substitution. In Apo A-1 Carnivora/Sapien Polypeptide 1 the amino acid glutamine (Gln196) is replaced by the amino acid glutamic acid (Glu196). More specifically, a Glutamine (Gln, Q) at position #196 in the wild-type human Apo A-1 preprotein amino acid sequence Apo A-1 (SEQ ID NO: 1) is substituted with a Glutamic Acid (Glu, E) (i.e., “Q to E at #196”) in the Apo A-1 Carnivora/Sapien Polypeptide 1 amino acid sequence as shown by SEQ ID NO: 2.

According to a first aspect of the invention, isolated polynucleotides are provided which encode amino acid sequences corresponding to Apo A-1 Carnivora/Sapien polypeptides.

Another form of the invention provides polypeptides corresponding to isolated DNA molecules. In certain embodiments, the amino acid sequences of the corresponding encoded polypeptides are shown for Apo A-1 Carnivora/Sapien Polypeptide 1 as SEQ ID NO: 2.

In one aspect, the present invention provides compositions and methods for the treatment of atherosclerosis or related conditions by administering a composition comprising synthetic Apo A-1 Carnivora/Sapien polypeptides, or other biologically active variants thereof. Apo A-1 Carnivora/Sapien are novel synthetic variants of the wild-type apolipoprotein, Apo A-1. Subjects treated with Apo A-1 Carnivora/Sapien polypeptides are advantageously benefited by reduction in atheromatous plaques and/or decreased HDL cholesterol.

Apolipoprotein A-1 Carnivora/Sapien polypeptides are identified as novel variants of wild-type human Apolipoprotein A-1 polypeptide that comprise one or more amino acid substitutions, or combinations thereof, selected from a group of Carnivora consensus amino acids substitutions consisting of: V to L at #77, F to V at #81, S to T at #82, L to I at #88, G to V at #105, A to Q at #119, M to V at #136, E to A at #144, R to G at #147, Q to R at #151, H to Q at #159, M to L at #172, A to T at #178, T to A at #185, H to Q at #186, Q to E at #196, E to Q at #203, N to G at #208, R to S at #212, T to S at #221, T to A at #226, F to L at #253, S to A at #255, L to V at #257, E to D at #258, Y to A at #260, and T to A at #266.

The numbers following the “#” symbols in the above group denote each specific location of an amino acid where the substitution is identified in the wild-type human Apo A-1 preprotein amino acid sequence.

Consistent with one aspect of the invention, as an illustrative example, one embodiment provides an amino acid sequence corresponding with an Apo A-1 Carnivora/Sapien polypeptide which is identical in amino acid sequence to that of wild-type human Apo A-1 except for the amino acid at position #77, which is a Leucine (L, Leu) instead of a Valine (V, Val).

Conventional methods of synthetic or recombinant production and subsequent purification techniques are well-known in the art for Apo A-1 polypeptides and may be readily applied to the methods of production of Apo A-1 Carnivora/Sapien polypeptides.

The nucleotide sequence coding for a A-1 Carnivora/Sapien or variants thereof can be inserted into an appropriate expression vector, i.e., a vector which contains the necessary elements for the transcription and translation of the inserted protein-coding sequence. The necessary transcriptional and translational signals can also be supplied by a native human wild-type A-1 gene and/or its flanking regions. A variety of host-vector systems may be utilized to express the protein-coding sequence. These include but are not limited to mammalian cell systems infected with a recombinant virus (e.g., vaccinia virus, adenovirus, etc.); insect cell systems infected with a recombinant virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Any of the methods previously described for the insertion of DNA fragments into a vector may be used to construct expression vectors containing a chimeric gene consisting of appropriate transcriptional/translational control signals and the protein coding sequences. These methods may include in vitro recombinant DNA and synthetic techniques. Expression of a nucleic acid sequence encoding a A-1 Carnivora/Sapien polypeptide may be regulated by a second nucleic acid sequence so that the A-1 Carnivora/Sapien is expressed in a host transformed with the recombinant DNA molecule. For example, expression of A-1 Carnivora/Sapien may be controlled by any promoter/enhancer element known in the art. Promoters that may be used to control expression of A-1 Carnivora/Sapien-encoding genes include, but are not limited to, the SV40 early promoter region (Bernoist and Chambon, Nature 290:304-310 (1981)), the promoter contained in the 3′ long terminal repeat of Rous sarcoma virus (Yamamoto et al, Cell 22:787-797 (1980)), the herpes thymidine kinase promoter (Wagner et al, Proc. Natl. Acad. Sci. USA. (1981) 78:1441-45), the regulatory sequences of the metallothionein gene (Brinster et al. Nature (1982) 296:39-42); prokaryotic expression vectors such as the 3-lactamase promoter (Villa-Kamaroff et al, Proc. Natl. Acad. Sci. USA. (1978) 75:3727-31), or the tat promoter (DeBoer et al, Proc. Natl. Acad. Sci. USA. (1983) 80:21-25).

A person of skill in the art will appreciate that cDNA, genomic, and synthesized sequences can be cloned and expressed. One way to accomplish such expression is by transferring a nucleic acid encoding an A-1 Carnivora/Sapien polypeptide or variant thereof, to cells in tissue culture. In addition to transferring a nucleic acid comprising a nucleic acid sequence encoding an A-1 Carnivora/Sapien polypeptide, the transferred nucleic acids can encode a functional portion of a particular A-1 Carnivora/Sapien polypeptide, or a protein having at least 60% sequence identity to an A-1 Carnivora/Sapien polypeptide disclosed herein, as compared over the length of the particular A-1 Carnivora/Sapien polypeptide, or a polypeptide having at least 60% sequence similarity to an A-1 Carnivora/Sapien variant, as compared over the length of the A-1 Carnivora/Sapien variant. Introduction of the nucleic acid into the cell may be accomplished by such methods as electroporation, lipofection, calcium phosphate mediated transfection, or viral infection. Usually, the method of transfer includes the transfer of a selectable marker (e.g., an antibiotic resistance gene) to the cells. The cells are then placed under selection to isolate those cells that have taken up and are expressing the transferred DNA.

The polynucleotides of the invention are either RNA or DNA (cDNA, genomic DNA, or synthetic DNA), or modifications, variants, homologues or fragments thereof. The DNA is either double-stranded or single-stranded, and, if single-stranded, is either the coding strand or the non-coding (antisense) strand. Any one of the sequences that encode the polypeptides of the invention, such as the sequence identified as SEQ ID NO: 2, may be (a) a coding sequence, (b) a ribonucleotide sequence derived from transcription of (a), or (c) a coding sequence which uses the redundancy or degeneracy of the genetic code to encode the same polypeptides. By “polypeptide” or “protein” is meant any chain of amino acids, regardless of length or post-translational modification (e.g., glycosylation or phosphorylation). Both terms are used interchangeably in the present application.

A further aspect of the invention provides amino acid sequences that are homologous to any one of the Apo A-1 Carnivora/Sapien polypeptides. The homologous amino acid sequence is any polypeptide that may be encoded, in whole or in part, by a nucleic acid sequence which hybridizes at 25-35 degrees Celsius below critical melting temperature (Tm), to any portion of the nucleic acid sequences of the Apo A-1 Carnivora/Sapien polypeptides. A homologous amino acid sequence is one that differs from an amino acid sequence shown in any one of the Apo A-1 Carnivora/Sapien polypeptides by one or more conservative amino acid substitutions. Such a sequence also encompass slight variants as well as sequences containing deletions or insertions that retain inherent characteristics of the polypeptide such as atheromatous plaque reduction. Preferably, such a sequence is at least 85%, 90%, or 95% identical to any one of the Apo A-1 Carnivora/Sapien polypeptides.

Homologous amino acid sequences include sequences that are identical or substantially identical to SEQ ID No: 2. By “amino acid sequence substantially identical” is meant a sequence that is at least 90% identical to a reference amino acid sequence and that preferably differs from the sequence of reference by a majority of conservative amino acid substitutions. In certain embodiments, the amino acid sequence may be 95%, 97%, or 99% identical to a reference amino acid sequence such as SEQ ID NO: 2.

Conservative amino acid substitutions are substitutions among amino acids of the same class. These classes include, for example, amino acids having uncharged polar side chains, such as asparagine, glutamine, serine, threonine, and tyrosine; amino acids having basic side chains, such as lysine, arginine, and histidine; amino acids having acidic side chains, such as aspartic acid and glutamic acid; and amino acids having nonpolar side chains, such as glycine, alanine, valine, leucine, isoleucine, proline, phenylalanine, methionine, tryptophan, and cysteine.

Homology may be measured using sequence analysis software. Sequence analysis software is well-known by those skilled in the art. Amino acid sequences are aligned to maximize identity. Gaps may be artificially introduced into the sequence to attain proper alignment. Once the optimal alignment has been set up, the degree of homology is established by recording all of the positions in which the amino acids of both sequences are identical, relative to the total number of positions.

Homologous polynucleotide sequences are defined in a similar way. A homologous sequence is one that is at least 45% identical to any one of coding sequences corresponding to the amino acid sequences of the Apo A-1 Carnivora/Sapien polypeptides. In certain embodiments, the homologous sequences are 60% or 85% identical to any one of coding sequences corresponding to the amino acid sequences of the Apo A-1 Carnivora/Sapien polypeptides.

Consistent with certain aspects of the invention, polypeptides having a sequence homologous to any one of the Apo A-1 Carnivora/Sapien polypeptides include naturally-occurring allelic variants, as well as mutants or any other non-naturally occurring variants that retain the inherent characteristics of the Apo A-1 Carnivora/Sapien polypeptides. For example, a modified Apo A-1 Carnivora/Sapien Polypeptide 1 which is modified by way of an additional glutamine at the end (i.e., Gln267) is homologous to Carnivora/Sapien Polypeptide 1 as identified by SEQ ID No 2.

As is known in the art, an allelic variant is an alternate form of a polypeptide that is characterized as having a substitution, deletion, or addition of one or more amino acids that does not alter the biological function of the polypeptide. By “biological function” is meant the function of the polypeptide in the cells in which it naturally occurs, even if the function is not necessary for the growth or survival of the cells. For example, a biological function of Apo A-1 is to act as a cofactor for lecithin cholesterolacyltransferase (LCAT) in the formation of plasma cholesteryl esters. A polypeptide can have more than one biological function.

Allelic variants are very common in nature. For example, the human species may be represented as a variety of populations that differ from each other by minor allelic variations. Indeed, a polypeptide that fulfills the same biological function in different populations can have an amino acid sequence (and polynucleotide sequence) that is not identical in each of the populations.

Useful homologs and fragments thereof that do not occur naturally are designed using known methods for identifying regions of a polypeptide that are likely to tolerate amino acid sequence changes and/or deletions and still exhibit a biological function. As an example, homologous polypeptides from different species are compared; conserved sequences are identified. The more divergent sequences are the most likely to tolerate sequence changes.

Consistent with the other aspects of the invention are polypeptides, homologs or fragments that are modified or treated to enhance their activity in the target animal or humans, in which the polypeptide, homolog or fragments are intended to confer protection against an autoimmune or inflammatory condition. Such modifications or treatments include: amino acid substitutions with an amino acid derivative such as 3-methyhistidine, 4-hydroxyproline, 5-hydroxylysine etc., modifications or deletions which are carried out after preparation of the polypeptide, homolog or fragment, such as the modification of free amino, carboxyl or hydroxyl side groups of the amino acids.

Homology among sequences may be analyzed using homology-searching algorithms. An alternative is to mutate a particular amino acid residue or sequence within the polypeptide in vitro, and then screen the mutant polypeptides for their ability to prevent or treat an atherosclerosis condition according to the method outlined below.

A person skilled in the art will readily understand that by following the screening process of this invention, it can be readily determined whether a particular homolog or fragment of any Apo A-1 Carnivora/Sapien polypeptide may be useful in the prevention or treatment of atherosclerosis. In certain embodiments, the screening procedure comprises the steps:

(i) selecting an animal model previously demonstrated to have atherosclerosis susceptibility, preferably a rabbit (e.g., the snowshoe hare (Lepus americanus));
(ii) immunizing the animal with the test homolog or fragment;
(iii) providing the immunized animal with an atherosclerosis-inducing diet (e.g., a diet containing 1% cholesterol); and,
(iv) selecting those homologs or fragments that confer protection against atherosclerosis.

Animal models for atherosclerosis are well known in the art and are described by DR Gross (D R Gross, Animal Models in Cardiovascular Research 3rd ed., Springer (2009) New York, N.Y.).

By “conferring protection” is meant that there is a reduction in severity of the condition in comparison with a control animal which was not provided with the test homolog or fragment.

“Protection” is defined such that individuals with protection will either avoid the condition entirely or show halted progression or recovery from the condition. For example, an individual who has protection against atherosclerosis may show clearance of atheromatous plaques. Individuals who lack protection can be susceptible to developing the disease.

A further aspect of the invention provides screening processes to identify additional variants of a polypeptide useful in the prevention or treatment of atherosclerosis. A person skilled in the art will readily understand that by following the screening process of this invention, it will be determined without undue experimentation whether a particular polypeptide, or homolog or fragment thereof, may be useful in the prevention or treatment of atherosclerosis. In certain embodiments, the screening procedure comprises the steps:

(i) identifying an amino acid sequence corresponding to an Apo A-1 polypeptide of an animal of the Carnivora order or other animal that is known to be resistant to atherosclerosis;
(ii) aligning the sequence with known amino acid sequences of wild-type human Apo A-1 and of Apo A-1 of other animals of the Carnivora order and/or other animals that is known to be resistant to atherosclerosis;
(iii) observing consensus or partial consensus amino acids among the aligned regions excluding the wild-type human Apo A-1;
(iv) contrasting the identified consensus or partial consensus amino acids with known amino acid sequences of the wild-type human Apo A-1 to identify differences;
(v) selecting the majority amino acid of the consensus or partial consensus amino acid that does has a contrasting amino acid (i.e., a non-identical amino acid in same alignment location of the wild-type human Apo A-1);
(vi) producing a synthetic test polypeptide comprising a wild-type human Apo A-1 modified with a substitution of its contrasting amino acid by the selected amino acid; and
(vii) selecting an animal previously demonstrated to have atherosclerosis susceptibility, preferably a rabbit (e.g., the snowshoe hare (Lepus americanus));
(viii) immunizing the animal, with the test polypeptide or homolog or fragment thereof;
(ix) inoculating the immunized animal with an atherosclerosis-inducing diet (e.g., diet containing 1% cholesterol); and,
(x) selecting those polypeptides or homologs or fragments thereof which confer protection against atherosclerosis.

Steps (i) to (v) of the above screening process may be performed using conventional sequence analysis software. Polypeptide data and sequence analysis tools are well known in the art and are readily available by accessing UniProt and its related features. UniProt is a comprehensive protein resource that is well known in the art and is a central repository of protein data created by combining other protein resources and managed by the UniProt Consortium comprising the European Bioinformatics Institute (EBI), the Swiss Institute of Bioinformatics (SIB), and the Protein Information Resource (PIR). EBI is located at the Wellcome Trust Genome Campus in Hinxton, UK, SIB is located in Geneva, Switzerland. PIR is hosted by the National Biomedical Research Foundation (NBRF) (Protein Information Resource, Georgetown University Medical Center, 3300 Whitehaven Street NW, Suite 1200, Washington, D.C. 20007).

The polypeptides of the present invention are useful for preventing and treating atherosclerosis and related diseases, and thus are useful for the manufacture of pharmaceutical compositions which contain an effective amount of an A1 CS polypeptide and/or A1 CS-rAAV vector in admixture with inorganic or organic, solid or liquid, pharmaceutically acceptable carriers. Thus, another aspect of this invention is a composition for preventing and treating atherosclerosis and related diseases, described herein in combination with a pharmaceutically acceptable excipient.

The pharmaceutical compositions according to the invention are those which are suitable for oral, transdermal, topical, or parenteral, such as intramuscular or intravenous, administration to humans, and which contain one or more polypeptides of the present invention together with a pharmaceutically acceptable carrier. The dosage depends on various factors such as the age, weight, severity of vascular condition, and other factors a physician or other qualified healthcare provider might identify.

In various embodiments, the therapeutic compositions are administered via suppository, or in tablet or capsule formulations for oral delivery. Oral formulations usually include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, enterics, sustained release formulations, powders, and the like.

Oral formulations for gene therapy are known in the art and are described by Chen et al. (Chen et al. World J. Gastroenterol (2004) 10(1)112-16). Further, other oral formulations are contemplated for use in the present invention as will be recognized by one of skill in the art.

Additional formulations which are suitable for other modes of administration, such as transdermal and topical administration, include salves, tinctures, creams, lotions, transdermal patches, transplanted skin, genetically engineered skin, stent coatings and suppositories. For salves and creams, traditional binders, carriers and excipients may include, for example, polyalkylene glycols or triglycerides. In certain embodiments, a transdermal patch may be used for delivering therapeutics. See, e.g. U.S. Pat. No. 4,638,043. Transdermal and topical formulations for gene therapy are known in the art. See, e.g. Jensen, T G (2004) Expert Opin Biol Ther. 4(5):677-82. Further, other transdermal and topical formulations are contemplated for use in the present invention as will be recognized by one of skill in the art.

Additional methods for administering peptides and proteins to indiviudals are disclosed, for example, in Antosova et al. (2009) Trends in Biotechnology 27(11):628-35.

Particularly suitable dosage forms for parenteral administration are sterile aqueous solutions of polypeptides of the present invention in water-soluble form, for example, a water-soluble salt, or sterile aqueous injection suspensions which contain substances increasing the viscosity, for example, sodium, carboxymethyl cellulose, sorbitol and/or dextran, and optionally stabilizers. In addition, polypeptides of the present invention, with or without adjuvants, can also be in lyophilized form and brought into solution prior to parenteral administration by the addition of suitable solvents.

Generally, an injectable composition of the invention may be a solution that is ready for injection, or a dry soluble composition that is ready to be combined with a solvent just prior to use, or a liquid concentrate ready for dilution prior to administration. In preparing a composition for injection strict attention must be paid to tonicity adjustment to avoid irritation.

The vehicle normally has no therapeutic activity and is nontoxic, but presents the polypeptides of the present invention to the body tissues or circulation in a form appropriate for absorption. Absorption normally will occur most rapidly and completely when the polypeptides of the present invention are presented as an aqueous solution. However, modification of the vehicle with water-miscible liquids or substitution with water-immiscible liquids can affect the rate of absorption. In preparing the compositions which are suitable for subcutaneous injection, one can use aqueous vehicles, water-miscible vehicles, and nonaqueous vehicles. Certain aqueous vehicles are well known for use in parenterals generally.

Water-miscible vehicles are also useful in the formulation of a parenteral composition of this invention. These solvents are used primarily to affect the solubility of peptides of the current invention. These solvents may include, for example, ethyl alcohol, polyethylene glycol and propylene glycol.

Additional substances may be included in the injectable compositions of this invention to improve or safeguard the quality of the composition. An added substance may, for example, affect solubility, provide for patient comfort, enhance the chemical stability, or protect preparation against the growth of microorganisms. Thus, the composition may include an appropriate solubilizer, substances to make a solution isotonic, substances to act as antioxidants, and substances that act as a preservative to prevent the growth of microorganisms. These substances will be present in an amount that is appropriate for their function, but will not adversely affect the action of the composition as a treatment for disease conditions as contemplated herein.

Generally, a sterile, parenterally injectable composition of this invention and other therapeutic formulations suitable for delivery to a mammal in accordance with various embodiments of the present invention can be readily prepared by routine experimentation by the skilled artisan. Guidance as to suitable pharmaceutical formulations are provided by Remington: The Science and Practice of Pharmacy 21st Ed. (2005) Lippincott Williams & Wilkins publishers.

Therapeutic and prophylactic efficacy may be evaluated using standard methods in the art, e.g., by measuring blood perfusion and/or atheromatous plaque size, using, e.g., a mouse model. Those skilled in the art will readily recognize that the particular model may be replaced with another model (e.g., rabbit model). For example, the efficacy of the polypeptides of the invention may be evaluated in an Apolipoprotein E knockout (Apoe−/−) mouse model. Apoe−/− mice have decreased serum apolipoprotein E and exhibit lipid abnormalities and atherosclerosis at a young age even when the mice are fed a diet low in cholesterol.

Protection is determined by comparing the degree of atherosclerosis to that of a control group. Protection is shown when atherosclerosis is reduced by comparison to the control group. Such an evaluation may be made for polynucleotides of the present invention.

A further aspect of the invention provides a modified Apo A-1 Carnivora/Sapien Polypeptides comprising an additional amino acid substitution, found in nature in a human Apo A-1 variant (described as human Apo A-1 Milano), which advantageously reduces the incidence of atherosclerosis. The modified polypeptide has the additional substitution of the amino acid arginine (Arg197) replaced by the amino acid cysteine (Cys197). For the example, a modified version of Apo A-1 Carnivora/Sapien Polypeptide 1 (SEQ ID NO: 2) comprises the amino sequence of the wild-type human Apo A-1 modified by two amino acid substitutions. One of the two substitutions is the amino acid glutamine (Gln196) replaced by the amino acid glutamic acid (Glu196). The other substitution, identified at the amino acid adjacent to the first substitution, is the amino acid arginine (Arg197) replaced by the amino acid cysteine (Cys197).

A further aspect of the invention provides methods and compositions for an apolipoprotein A-1 Carnivora/Sapien (A1 CS) or a apolipoprotein A-1 Carnivora/Sapien-lipid complex for use in the treatment, amelioration or prevention of age-related macular degeneration (ARMD), including exudative (Wet ARMD) and non-exudative (dry ARMD) types. Lipid compositions, methods of administration, and therapeutic dosing regimens for lipoprotein-lipid complexes for use in treatment of ARMD are well known in the art (e.g., U.S. patent application Ser. No. 11/707,809) and may be readily applied to the polypeptide-lipid complexes of the present invention.

A further embodiment of the present invention provides a method of treating atherosclerosis or related disorders in a subject in need thereof, said method comprising administering an Apolipoprotein A-1 Carnivora/Sapien (A1 CS) polypeptide or Apolipoprotein A-1 Carnivora/Sapien:1-palmitoyl-2-oleoyl-sn-glycero-3-phosphocholine complex (A1 CS:POPC complex) at a dose of about 1 mg of protein/kg to about 100 mg of protein/kg.

A further embodiment provides a method of treating atherosclerosis or related disorders in a subject in need thereof, said method comprising administering an A1 CS polypeptide or A1 CS:POPC complex at a dose of about 15 mg/kg or 45 mg/kg.

A further embodiment provides a method of treating atherosclerosis or related disorders in a subject in need thereof, said method comprising administering an A1 CS polypeptide or A1 CS:POPC complex at a dose of about 15 mg/kg to about 45 mg/kg.

A further embodiment provides a method of treating atherosclerosis or related disorders in a subject in need thereof, said method comprising administering an A1 CS polypeptide or A1 CS:POPC complex wherein the Apo A-1 Carnivora/Sapien is recombinant Apo A-1 Carnivora/Sapien.

A further embodiment provides a method of treating atherosclerosis or related disorders in a subject in need thereof, said method comprising administering an A1 CS polypeptide or A1 CS:POPC complex wherein the Apo A1 CS:POPC complex is comprised of Apolipoprotein A-1 Carnivora/Sapien and 1-palmitoyl-2-oleoyl-sn-glycero-3-phosphocholine in a ratio of about 1:1 wt protein/wt lipid.

A further embodiment provides a method of treating atherosclerosis or related disorders in a subject in need thereof, said method comprising administering an Apo A1 CS polypeptide or Apo A1 CS:POPC complex wherein the Apo A1 CS polypeptide or Apo A1 CS:POPC complex is a sterile liquid pharmaceutical formulation.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation is administered to a subject at a dose of about 15 mg/kg or 45 mg/kg.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation is administered to a subject once weekly for about 6 months, about 5 months, about 4 months, about 3 months, about 2 months, or about 1 month.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation is administered to a subject about every day for about 1 month.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation is administered to a subject about every 3 days for about 1 month to about 6 months.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation is administered about every 10 days for about 1 month to about 6 months.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation is administered about every 14 days for about 1 month to about 6 months.

A further embodiment provides a method of treating acute coronary syndromes or related disorders in a subject in need thereof, said method comprising administering an A1 CS polypeptide or A1 CS:POPC complex, and further comprising surgical intervention.

A further embodiment provides a method of treating acute coronary syndromes or related disorders in a subject in need thereof, said method comprising administering an A1 CS polypeptide or A1 CS:POPC complex, and further comprising surgical intervention, wherein the surgical intervention comprises percutaneous transluminal coronary angioplasty or coronary artery bypass grafting.

A further embodiment provides a method of treating atherosclerosis or related disorders in a subject in need thereof, said method comprising administering an A1 CS polypeptide or A1 CS:POPC complex, and further comprising administration of another drug to treat, prevent or ameliorate the diseases, disorders, symptoms or pain associated with atherosclerosis.

A further embodiment provides a method of treating atherosclerosis or related disorders in a subject in need thereof, said method comprising administering an A1 CS polypeptide or A1 CS:POPC complex, wherein the percent atheroma volume in a subject's affected vessel is reduced.

A further embodiment provides a method of treating atherosclerosis or related disorders in a subject in need thereof, said method comprising administering an A1 CS polypeptide or A1 CS:POPC complex, wherein the total atheroma volume in the target vessel of the subject is reduced.

A further embodiment provides a method of treating atherosclerosis or related disorders in a subject in need thereof, said method comprising administering an A1 CS polypeptide or A1 CS:POPC complex, wherein the A1 CS polypeptide or A1 CS:POPC complex, wherein the mean maximum atheroma thickness in the subject's target coronary segment is reduced.

A further embodiment provides a method of treating atherosclerosis or related disorders in a subject in need thereof, said method comprising administering A1 CS polypeptide formulation or A1 CS:POPC complex, wherein the A1 CS polypeptide formulation or A1 CS:POPC complex is a sterile liquid, and wherein the pharmaceutical formulation is administered intravenously.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation is administered intravenously, and wherein the pharmaceutical formulation is administered over about one hour.

A further embodiment provides a method of treating atherosclerosis or related disorders in a subject in need thereof, said method comprising administering A1 CS polypeptide formulation or A1 CS:POPC complex, wherein the A1 CS polypeptide formulation or A1 CS:POPC complex is a sterile liquid, wherein the pharmaceutical formulation is administered intravenously, and wherein the pharmaceutical formulation is administered over about three hours.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation comprises an Apo A1 CS polypeptide or A1 CS:POPC complex, a sucrose-mannitol carrier and a phosphate buffer.

A further embodiment provides a method of treating atherosclerosis or related disorders in a subject in need thereof, said method comprising administering A1 CS polypeptide formulation or A1 CS:POPC complex, wherein the A1 CS polypeptide formulation or A1 CS:POPC complex is a sterile liquid, wherein the pharmaceutical formulation comprises Apo A1 CS, POPC, a sucrose-mannitol carrier and a phosphate buffer, and wherein the sucrose-mannitol carrier consists of about 6.0% to about 6.4% sucrose and about 0.8% to about 1% mannitol.

A further embodiment provides a method of treating atherosclerosis or related disorders in a subject in need thereof, said method comprising administering A1 CS polypeptide formulation or A1 CS:POPC complex, wherein the A1 CS polypeptide formulation or A1 CS:POPC complex is a sterile liquid, wherein the pharmaceutical formulation comprises Apo A1 CS, POPC, a sucrose-mannitol carrier and a phosphate buffer, and wherein the sucrose-mannitol carrier consists of about 6.2% sucrose and about 0.9% mannitol.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, and wherein the pharmaceutical formulation has a pH of about 7.0 to about 7.8.

A further embodiment provides a method of treating atherosclerosis or related disorders in a subject in need thereof, said method comprising administering A1 CS polypeptide formulation or A1 CS:POPC complex, wherein the A1 CS polypeptide formulation or A1 CS:POPC complex is a sterile liquid and the pH is about 7.5.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation comprises about 12 mg/ml to about 18 mg/ml Apo A1 CS.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation comprises about 11 mg/ml to about 17 mg/ml of POPC.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation comprises less than 6,000 particulates greater than 10 μm in size per 50 mL.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation comprises less than 600 particulates greater than 25 μm in size per 50 mL.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation has an Osmolality of about 280 to about 320 mOsm.

A further embodiment provides a method of treating atherosclerosis or related disorders in a subject in need thereof, said method comprising administering an A1 CS polypeptide formulation or A1 CS:POPC complex, wherein the A1 CS polypeptide formulation or A1 CS:POPC complex is a sterile liquid, wherein the pharmaceutical formulation has an Osmolality of about 290 mOsm.

A further embodiment of the present invention provides a method of reducing atherosclerotic plaque in a subject in need thereof, said method comprising administering 45 mg/kg of recombinant A1 CS polypeptide or Apo A1 CS:POPC complex to the subject at least once.

A further embodiment of the present invention provides a method of reducing atherosclerotic plaque in a subject in need thereof, said method comprising administering 45 mg/kg of recombinant A1 CS polypeptide or Apo A1 CS:POPC complex to the subject at least once, wherein the recombinant A1 CS polypeptide or Apo A1 CS:POPC complex is administered weekly, monthly or annually.

A further embodiment of the present invention provides a method of reducing atherosclerotic plaque in a subject in need thereof, said method comprising administering 45 mg/kg of recombinant A1 CS polypeptide or Apo A1 CS:POPC complex to the subject at least once, wherein the recombinant A1 CS polypeptide or Apo A1 CS:POPC complex is administered weekly for about 3-5 weeks.

A further embodiment of the present invention provides a method of reducing atherosclerotic plaque in a subject in need thereof, said method comprising administering 45 mg/kg of recombinant A1 CS polypeptide or Apo A1 CS:POPC complex to the subject at least once, further comprising administering to the subject a dose of 15 mg/kg of recombinant A1 CS polypeptide or Apo A1 CS:POPC complex at least once.

A further embodiment of the present invention provides a method of reducing atherosclerotic plaque in a subject in need thereof, said method comprising administering 45 mg/kg of recombinant A1 CS polypeptide or Apo A1 CS:POPC complex to the subject at least once, further comprising administering to the subject a dose of 15 mg/kg of recombinant A1 CS polypeptide or Apo A1 CS:POPC complex at least once, wherein the complex is administered weekly, monthly or annually.

A further embodiment of the present invention provides a method of reducing atherosclerotic plaque in a subject in need thereof, said method comprising administering 45 mg/kg of recombinant A1 CS polypeptide or Apo A1 CS:POPC complex to the subject at least once, wherein the subject is also treated with another drug to treat, prevent or ameliorate the diseases, disorders, symptoms or pain associated with acute coronary syndromes.

A further embodiment of the present invention provides a method of reducing atherosclerotic plaque in a subject in need thereof, said method comprising administering 45 mg/kg of recombinant A1 CS polypeptide or Apo A1 CS:POPC complex to the subject at least once, wherein the subject is also treated with another drug to treat, prevent or ameliorate the diseases, disorders, symptoms or pain associated with acute coronary syndromes, wherein the drug is a statin, β-blocker, ACE inhibitor, antithrombotic, vasodilator or combination thereof.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, comprising about 6.2% sucrose.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, comprising about 0.9% mannitol.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, wherein the pH is about 7.5.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, wherein the concentration of recombinant Apo A1 CS or variant thereof is about 12 mg per ml to about 18 mg per ml of said buffer.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, wherein the recombinant Apo A1 CS is a conservatively substituted Apo A1 CS.

A further embodiment of the present invention provides a liquid pharmaceutical formulation comprising a recombinant Apo A1 CS or variant thereof/POPC complex in a buffer, said buffer comprising about 6.0% to about 6.4% sucrose, further comprising about 0.8 to about 1% mannitol, said buffer having a pH at about 7.0 to about 7.8, wherein the recombinant Apo A1 CS and POPC are complexed in a ratio of 1:0.95 recombinant Apo A1 CS:POPC (wt protein/wt lipid).

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, comprising less than 6,000 particulates greater than 10 μm in size per 50 mL.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, comprising less than 600 particulates greater than 25 μm in size per 50 mL.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, wherein the Osmolality is about 280 to about 320 mOsm.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, wherein the Osmolality is about 280 to about 320 mOsm, wherein the Osmolality is about 290 mOsm.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, wherein the liquid pharmaceutical formulation is sterile.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, that is frozen.

A further embodiment of the present invention provides a liquid pharmaceutical formulation comprising a recombinant Apo A1 CS or variant thereof, alone or complexed with POPC in a buffer, said buffer comprising about 6.0% to about 6.4% sucrose, further comprising about 0.8 to about 1% mannitol, said buffer having a pH at about 7.0 to about 7.8, that is in a sterile vial, sterile pre-filled bag or pre-filled syringe.

A further embodiment of the present invention provides a pharmaceutical formulation comprising a recombinant Apo A1 CS, or variant thereof, alone or complexed with PO PC, and a phosphate buffer having a pH of about 7.0 to about 7.8, said buffer comprising 2% glucose and 4 mM of sodium phosphate.

A further embodiment of the present invention provides a pharmaceutical formulation comprising a recombinant Apo A1 CS, or variant thereof, alone or complexed with PO PC, and a phosphate buffer having a pH of about 7.0 to about 7.8, said buffer comprising 2% glucose and 4 mM of sodium phosphate, comprising phosphate buffer to achieve a pH of about 7.5.

A further embodiment of the present invention provides a pharmaceutical formulation comprising a recombinant Apo A1 CS, or variant thereof, alone or complexed with PO PC, and a phosphate buffer having a pH of about 7.0 to about 7.8, said buffer comprising 2% glucose and 4 mM of sodium phosphate, wherein the recombinant Apo A1 CS is a conservatively substituted Apo A1 CS.

A further embodiment of the present invention provides a pharmaceutical formulation comprising a recombinant Apo A1 CS, or variant thereof, complexed with 1-palmitoyl-2-oleoyl-sn-glycero-3-phosphocholine, and a phosphate buffer having a pH of about 7.0 to about 7.8, said buffer comprising 2% glucose and 4 mM of sodium phosphate, wherein the recombinant Apo A1 CS and POPC are complexed in a ratio of 1:0.95 recombinant Apo A1 CS:POPC (wt protein/wt lipid).

A further embodiment of the present invention provides a pharmaceutical formulation comprising a recombinant Apo A1 CS, or variant thereof, alone or complexed with PO PC, and a phosphate buffer having a pH of about 7.0 to about 7.8, said buffer comprising 2% glucose and 4 mM of sodium phosphate, comprising less than 6,000 particulates greater than 10 μm in size per 50 mL.

A further embodiment of the present invention provides a pharmaceutical formulation comprising a recombinant Apo A1 CS, or variant thereof, alone or complexed with POPC, and a phosphate buffer having a pH of about 7.0 to about 7.8, said buffer comprising 2% glucose and 4 mM of sodium phosphate, comprising less than 600 particulates greater than 25 μm in size per 50 mL.

A further embodiment of the present invention provides a pharmaceutical formulation comprising a recombinant Apo A1 CS, or variant thereof, alone or complexed with PO PC, and a phosphate buffer having a pH of about 7.0 to about 7.8, said buffer comprising 2% glucose and 4 mM of sodium phosphate, wherein the Osmolality is about 280 to about 320 mOsm.

A further embodiment of the present invention provides a pharmaceutical formulation comprising a recombinant Apo A1 CS, or variant thereof, alone or complexed with PO PC, and a phosphate buffer having a pH of about 7.0 to about 7.8, said buffer comprising 2% glucose and 4 mM of sodium phosphate, wherein the Osmolality is about 290 mOsm.

A further embodiment of the present invention provides a pharmaceutical formulation comprising a recombinant Apo A1 CS, or variant thereof, alone or complexed with PO PC, and a phosphate buffer having a pH of about 7.0 to about 7.8, said buffer comprising 2% glucose and 4 mM of sodium phosphate, wherein the pharmaceutical formulation is sterile.

A further embodiment of the present invention provides a pharmaceutical formulation comprising a recombinant Apo A1 CS, or variant thereof, alone or complexed with PO PC, and a phosphate buffer having a pH of about 7.0 to about 7.8, said buffer comprising 2% glucose and 4 mM of sodium phosphate, wherein the pharmaceutical formulation is frozen.

A further embodiment of the present invention provides a pharmaceutical formulation comprising a recombinant Apo A1 CS, or variant thereof, alone or complexed with POPC, and a phosphate buffer having a pH of about 7.0 to about 7.8, said buffer comprising 2% glucose and 4 mM of sodium phosphate, wherein the pharmaceutical formulation is in a sterile vial, sterile pre-filled bag or pre-filled syringe.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, comprising about 6.2% sucrose.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, comprising about 0.9% mannitol.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, further comprising a buffer, said buffer having a pH at about 7.5.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, wherein the recombinant Apo A1 CS is a conservatively substituted Apo A1 CS.

A further embodiment provides a lyophilized pharmaceutical formulation comprising: (a) recombinant Apo A1 CS, or variant thereof, complexed with 1-palmitoyl-2-oleoyl-sn-glycero-3-phosphocholine; (b) about 6.0% to about 6.4% sucrose; and (c) about 0.8 to about 1% mannitol, wherein the concentration of POPC is about 11 mg/ml to about 17 mg/ml.

A further embodiment provides a lyophilized pharmaceutical formulation comprising: (a) recombinant Apo A1 CS, or variant thereof, complexed with POPC; (b) about 6.0% to about 6.4% sucrose; and (c) about 0.8 to about 1% mannitol, wherein the recombinant Apo A1 CS and POPC are complexed in a ratio of 1:0.95 recombinant Apo A1 CS:POPC (wt protein/wt lipid).

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, comprising less than 6,000 particulates greater than 10 μm in size per 50 mL.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, comprising less than 600 particulates greater than 25 μm in size per 50 mL.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, wherein the Osmolality is about 280 to about 320 mOsm.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, wherein the Osmolality is about 290 mOsm.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, that is sterile.

A further embodiment provides a lyophilized pharmaceutical formulation comprising: (a) recombinant Apo A1 CS, or variant thereof, alone or complexed with POPC; (b) about 6.0% to about 6.4% sucrose; and (c) about 0.8 to about 1% mannitol, that is in a sterile vial, sterile pre-filled bag or pre-filled syringe.

A further embodiment provides a method of treating ischemic reperfusion in a subject in need thereof, said method comprising administering an A1 CS polypeptide or Apo A1 CS:POPC complex at a dose of about 15 mg of protein/kg or 45 mg of protein/kg.

Unless defined otherwise, technical and scientific terms used herein have the same meaning as commonly understood by one of ordinary skill in the art to which this invention belongs. Singleton et al., Dictionary of Microbiology and Molecular Biology 2nd ed, J. Wiley & Sons (New York, N.Y. 1994); March, Advanced Organic Chemistry Reactions, Mechanisms and Structure 4th ed., J. Wiley & Sons (New York, N.Y. 1992); Sambrook and Russel, Molecular Cloning: A Laboratory Manual 3rd ed., Cold Spring Harbor Laboratory Press (Cold Spring Harbor, N.Y. 2001); and U.S. patent application Ser. Nos. 10/599,692, 12/256,822, 11/707,809 and 10/967,061, provide one skilled in the art with a general guide to many of the terms used in the present application.

### Example

The invention will be further described with references to the following example. The example is intended only to be illustrative, but not to limit the scope of the invention in any sense.

### Background

A study is conducted on a standard atherosclerosis animal modal (preferably an apoE-deficient mouse model) testing whether a single high dose of recombinant Apo A-1 Carnivora/Sapien-phospholipid complex (A1 CS-I(m)) could rapidly mobilize tissue cholesterol and reduce plaque lipid and macrophage content in apoE-deficient mice.

### Methods

High cholesterol-fed, 26-week-old apoE-deficient mice receive either a single intravenous injection of saline or 1 000 milligram per kilogram (mg/kg) dipalmitoylphosphatidylcholine (DPPC) or 400 mg/kg of recombinant A1 CS-I(m) complexed with DPPC (1:2.7 weight ratio). Mouse blood is sampled before and at 1 hour and at 48 hours after initial injection. Aortic root plaques are evaluated for lipid content and macrophage content after oil-red O and immunostain procedure, respectively. One hour after the injection, the plasma cholesterol efflux-promoting capacity is significantly higher in recombinant A1 CS-I(m)-treated mice compared with saline-treated mice and DPPC-treated mice. The blood sampling and quantitative analysis occurs at 1 hour. The blood sampling and quantitative analysis occurs again at 48 hours.

### Results

The serum free cholesterol, as an index of tissue cholesterol mobilization, is compared with baseline values at 1 hour after recombinant A1 CAS-I(m) injection and is found to be significantly elevated. The serum free cholesterol remains significantly elevated at 48 hours. Mice receiving recombinant A1 CS-I(m) have significantly lower lipid content and macrophage content in their plaques when compared with the saline-treated mice and when compared with DPPC-treated mice. A single high dose of recombinant A1 CS-I(m) rapidly mobilizes tissue cholesterol and reduces plaque lipid and macrophage content in the mice. Repeated doses of recombinant A1 CS-I(m) reduce atherosclerosis and favorably change plaque composition in rabbits and mice.

Many modifications and variations of this invention can be made without departing from its spirit and scope, as will be apparent to those skilled in the art. The specific embodiments described herein are offered by way of example only.

All citations herein are incorporated herein by reference to the same extent as if each individual publication or patent document was specifically and individually indicated to be incorporated by reference. However, citation herein of any publication or patent document is not intended as an admission that the cited reference is pertinent prior art, nor does it constitute any admission as to the contents or effective prior art date of the reference.

1. A polypeptide comprising amino acids 25-266 of SEQ ID NO: 1, wherein the polypeptide further comprises one or more amino acid substitutions selected from the following: V to L at #77, F to V at #81, S to T at #82, L to I at #88, G to V at #105, A to Q at #119, M to V at #136, E to A at #144, R to G at #147, Q to R at #151, H to Q at #159, M to L at #172, A to T at #178, T to A at #185, H to Q at #186, Q to E at #196, E to Q at #203, N to G at #208, R to S at #212, T to S at #221, T to A at #226, F to L at #253, S to A at #255, L to V at #257, E to D at #258, Y to A at #260, and T to A at #266.

2. The polypeptide of claim 1, comprising amino acids 1-267 of SEQ ID NO: 1.

3. The polypeptide of claim 1, wherein the polypeptide comprises the amino acid substitution V to L at #77.

4. The polypeptide of claim 1, wherein the polypeptide comprises the amino acid substitution F to V at #81.

5. The polypeptide of claim 1, wherein the polypeptide comprises the amino acid substitution S to T at #82.

6. The polypeptide of claim 1, wherein the polypeptide comprises the amino acid substitution L to I at #88.

7. The polypeptide of claim 1, wherein the polypeptide comprises the amino acid substitution G to V at #105.

8. The polypeptide of claim 1, wherein the polypeptide comprises the amino acid substitution A to Q at #119.

9. The polypeptide of claim 1, wherein the polypeptide comprises the amino acid substitution M to V at #136.

10. The polypeptide of claim 1, wherein the polypeptide comprises the amino acid substitution E to A at #144.

11. The polypeptide of claim 1, wherein the polypeptide comprises the amino acid substitution R to G at #147.

12. The polypeptide of claim 1, wherein the polypeptide comprises the amino acid substitution Q to R at #151.

13. The polypeptide of claim 1, wherein the polypeptide comprises the amino acid substitution H to Q at #159.

14. The polypeptide of claim 1, wherein the polypeptide comprises the amino acid substitution M to L at #172.

15. The polypeptide of claim 1, wherein the polypeptide comprises the amino acid substitution A to T at #178.

16. The polypeptide of claim 1, wherein the polypeptide comprises the amino acid substitution T to A at #185.

17. The polypeptide of claim 1, wherein the polypeptide comprises the amino acid substitution H to Q at #186.

18. The polypeptide of claim 1, wherein the polypeptide comprises the amino acid substitution Q to E at #196.

19. The polypeptide of claim 1, wherein the polypeptide comprises the amino acid substitution E to Q at #203.

20. The polypeptide of claim 1, wherein the polypeptide comprises the amino acid substitution N to G at #208.

21. The polypeptide of claim 1, wherein the polypeptide comprises the amino acid substitution R to S at #212.

22. The polypeptide of claim 1, wherein the polypeptide comprises the amino acid substitution T to S at #221.

23. The polypeptide of claim 1, wherein the polypeptide comprises the amino acid substitution T to A at #226.

24. The polypeptide of claim 1, wherein the polypeptide comprises the amino acid substitution F to L at #253.

25. The polypeptide of claim 1, wherein the polypeptide comprises the amino acid substitution S to A at #255.

26. The polypeptide of claim 1, wherein the polypeptide comprises the amino acid substitution L to V at #257.

27. The polypeptide of claim 1, wherein the polypeptide comprises the amino acid substitution E to D at #258.

28. The polypeptide of claim 1, wherein the polypeptide comprises the amino acid substitution Y to A at #260.

29. The polypeptide of claim 1, wherein the polypeptide comprises the amino acid substitution T to A at #266.

30. A polynucleotide which codes for the polypeptide of claim 1, or the complement of which codes for the polypeptide of claim 1.

31. The polynucleotide of claim 30, further comprising a promoter element.

32. A method of treating an individual suffering from atherosclerosis or a related condition, comprising administering to the individual a therapeutically effective amount of a polypeptide comprising amino acids 25-266 of SEQ ID NO: 1, wherein the polypeptide further comprises one or more amino acid substitutions selected from the following: V to L at #77, F to V at #81, S to T at #82, L to I at #88, G to V at #105, A to Q at #119, M to V at #136, E to A at #144, R to G at #147, Q to R at #151, H to Q at #159, M to L at #172, A to T at #178, T to A at #185, H to Q at #186, Q to E at #196, E to Q at #203, N to G at #208, R to S at #212, T to S at #221, T to A at #226, F to L at #253, S to A at #255, L to V at #257, E to D at #258, Y to A at #260, and T to A at #266.

33. The method of claim 32, wherein the polypeptide is administered parenterally.

34. The method of claim 32, wherein the polypeptide is administered intravenously.

35. The method of claim 32, wherein the polypeptide is administered by injection.

36. A pharmaceutical composition comprising:
a polypeptide comprising amino acids 25-266 of SEQ ID NO: 1, wherein the polypeptide further comprises one or more amino acid substitutions selected from the following: V to L at #77, F to V at #81, S to T at #82, L to I at #88, G to V at #105, A to Q at #119, M to V at #136, E to A at #144, R to G at #147, Q to R at #151, H to Q at #159, M to L at #172, A to T at #178, T to A at #185, H to Q at #186, Q to E at #196, E to Q at #203, N to G at #208, R to S at #212, T to S at #221, T to A at #226, F to L at #253, S to A at #255, L to V at #257, E to D at #258, Y to A at #260, and T to A at #266; and
a pharmaceutically acceptable carrier.


SEQUENCE LISTING (2 sequences)

SEQ ID NO: 1 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 242 aa, Artificial Sequence
  Synthetic Apo A-I Carnivora/Sapien Polypeptide 1
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRERLAARLEALKENGGARLAEYHAKATEHLSTLSEKAKPALEDLRQGLLPVLESFKVSFLSALEEYTKKLNT